(19) United States
(12) Patent Application Publication
(10) Pub. No.: US 2022/0257434 A1
(43) Pub. Date: Aug. 18, 2022

(54) Tuck and Fold Diaper with Leak Protection

(71) Applicant: Carrie Small, Elm Grove, WI (US)

(72) Inventor: Carrie Small, Elm Grove, WI (US)

(21) Appl. No.: 17/672,374

(22) Filed: Feb. 15, 2022

Related U.S. Application Data
(60) Provisional application No. 63/150,382, filed on Feb. 17, 2021.

(51) Int. Cl. A61F 13/513; A61F 13/53; A61F 13/56; A61F 13/514

(52) U.S. Cl. CPC A61F 13/513; A61F 13/53; A61F 2013/530481; A61F 13/51496; A61F 2013/51388; A61F 13/5633

(57) ABSTRACT

A baby diaper includes an absorbent flap attached to the front panel and is foldable into the inside of the front top panel of the diaper, for example, tucked inside and around a baby boy's penis, to prevent baby boys from peeing out of the diaper and leaking onto their clothes and the environment. The absorbent flap extends along substantially an entire width of the front panel of the diaper to catch wetness along the entire frontal width and is preferably shaped to avoid irritation with the baby's legs.

### CROSS REFERENCE TO RELATED APPLICATION

This application claims the benefit of U.S. Provisional Patent Application 63/150,382, filed Feb. 17, 2021, which is incorporated by reference herein in its entirety.

### BACKGROUND OF THE INVENTION

The present invention relates generally to baby diapers for babies, newborns and infants and, in particular to a baby diaper for baby boys with an absorbent flap that tucks inside the front of a diaper to prevent leaks at the top front end of the diaper.

Diapers are typically worn by babies, newborns and infants before they are potty trained and by young children who experience bed wetting. The diapers absorb or contain the urine and waste to prevent the urine and waste products from soiling clothes or bedding.

Disposable diapers are generally made of superabsorbent polymers, resealable tapes, and elastic waist bands. The absorbent polymers are typically constructed in a layered construction that includes an innermost layer of nonwoven material that contacts the skin, a distribution layer which transfers wetness to an inner absorbent layer which may include air-laid paper and superabsorbent polymers, and an outermost layer of breathable polyethylene film or a nonwoven material and film composite that blocks wetness and soil transfer to the exterior environment. Adhesive tapes are typically used to fasten the diaper around the baby's waist. Elasticized fabric with extra layers of elastic barriers (i.e., single and double gussets) around the baby's legs and waist help to contain the waste products within the diaper, especially for excess wetness that has not been absorbed by the absorbent polymers.

Cloth diapers are similar to disposable diapers but are reusable and are made of natural fibers and/or synthetic materials. The diapers are typically fastened with hook and loop tape (i.e., Velcro) or snaps.

Diaper leaks, especially when the diaper is worn overnight, typically occur because there is not enough absorbent material to absorb the volume of urine, or there is a poor fit (i.e., the diaper is too big for the baby) which causes urine to leak out of gaps around the legs or waist instead of being absorbed by the absorbent material. Additionally, for baby boys, the direction of urine flow upward instead of downward may prevent urine from being absorbed by the absorbent material, or the urine may not even come into contact with the absorbent material, which may cause urine to flow up and out of the top front end of the diaper.

### SUMMARY OF THE INVENTION

The present invention provides an absorbent flap that is attached to, or is an extension of, the front top panel of a baby diaper and is foldable into the inside of the front panel of the diaper, for example, tucked inside and around a baby boy's penis to prevent baby boys from peeing out of the top front end of the diaper and leaking onto their clothes and the environment. The absorbent flap extends along substantially an entire width of the front panel of the diaper to catch wetness along the entire frontal width and is preferably shaped to avoid irritation with the baby's legs.

It is thus one feature of one embodiment of the present invention to catch and absorb excess urine from leaking out the top front end of the diaper at the baby's waist.

It is thus one feature of one embodiment of the present invention to tuck the flap around a baby boy's penis so that urine is caught and absorbed regardless of the direction of urine flow within the diaper.

One embodiment of the present invention provides a disposable diaper to be worn by a human baby and extending along a sagittal axis of the baby when worn, the diaper comprising an absorbent pad extending along the sagittal axis having at least one layer; a front panel extending from a front end of the absorbent pad and configured to extend along a front of a baby's lower torso and supporting a front waist region configured to extend along a baby's waist; a back panel extending from a rear end of the absorbent pad and configured to extend along a rear of a baby's lower torso and supporting a back waist region configured to extend along a baby's waist and to couple to the front waist region; and an absorbent sheath extending outwardly from the front panel and extendable along an inner surface of the front panel between the front panel and the front of the baby's lower torso when worn by the baby.

It is thus one feature of one embodiment of the present invention to catch and absorb urine that is directed upward toward the top front end of the diaper.

The absorbent sheath may be thinner than the absorbent pad, the thickness measured along a longitudinal axis perpendicular to the sagittal axis.

It is thus one feature of one embodiment of the present invention to provide a comfortable absorbent pad to a front of the diaper that can be easily tucked into the front of the front panel without discomfort.

The absorbent sheath may have a thickness that is less than 0.3 inches, the thickness measured along a longitudinal axis perpendicular to the sagittal axis.

It is thus one feature of one embodiment of the present invention to provide an unobtrusive and discreet absorbent pad to a front of the diaper.

The absorbent sheath may comprise an absorbent layer configured to absorb and trap liquid.

It is thus one feature of one embodiment of the present invention to provide additional absorbing material to the diaper to prevent leaks in areas of exposure.

The absorbing material may be comprised of at least one of a fibrous material and a super absorbing polymer.

It is thus one feature of one embodiment of the present invention to use quick and highly absorbent materials commonly used to absorb and retain large amounts of liquid.

The absorbent sheath may comprise a contacting layer that is configured to contact the baby's lower torso and is permeable to liquids.

It is thus one feature of one embodiment of the present invention to provide a dry contact surface that does not irritate the skin or cause skin rashes.

The absorbent sheath may be a flap attached at a front end of the front panel, or an extension of the front end of the front panel and is foldable downwardly toward a rear end of the front panel.

It is thus one feature of one embodiment of the present invention to position the flap around a baby boy's penis before or after the diaper is secured to the baby.

The absorbent sheath may extend substantially an entire width of a top edge of the front panel.

It is thus one feature of one embodiment of the present invention to provide a barrier to liquid escaping from the inside of the diaper at the top front end of the diaper.

The absorbent sheath may have a width of at least 3 inches. The absorbent sheath may have a width of at least 4 inches.

It is thus one feature of one embodiment of the present invention that the absorbent sheath extends across the entire top end of the front of the diaper.

The absorbent sheath may extend at least one-third a length of the front panel, the length measured along the longitudinal axis. The absorbent sheath may have a length of at least 2 inches. The absorbent sheath may have a length of at least 3 inches.

It is thus one feature of one embodiment of the present invention to tuck a baby boy's penis between the absorbent sheath and the front panel to catch upward urine flow.

The flap may be substantially rectangular. The edges of the flap may be curved.

It is thus one feature of one embodiment of the present invention to shape the flap so that it resembles the front panel and therefore does not interfere with (or irritate by rubbing) the baby's legs when worn.

The at least one layer of the absorbent substrate comprises an innermost layer that is configured to contact the baby's lower torso and is permeable to liquids, an interior layer that is configured to absorb and trap liquid, and an outermost layer that is configured to be exposed to the environment and is impermeable to liquid.

It is thus one feature of one embodiment of the present invention to provide expected absorbency of urine and waste within the diaper pad.

An outer surface of the front panel may include a graphic design.

It is thus one feature of one embodiment of the present invention to recognize a front of diaper easily visually.

Fasteners may extend outwardly from the back waist region of the rear side panel. The fasteners may include an adhesive. The front waist region and back waist region may be elastic.

It is thus one feature of one embodiment of the present invention to provide quick placement of the diaper on the baby and to secure the top of the diaper tightly around the baby's waist to prevent leaks.

An alternative embodiment of the present invention provides a method of putting an absorbent article on a baby including: providing a disposable diaper having an absorbent pad extending along the sagittal axis of the baby having at least one layer, a front panel extending from a front end of the absorbent pad and configured to extend along a front of a baby's lower torso and supporting a front waist region configured to extend along a baby's waist, a back panel extending from a rear end of the absorbent pad and configured to extend along a rear of a baby's lower torso and supporting a back waist region configured to extend along a baby's waist and to couple to the front waist region, and an absorbent sheath extending outwardly from the front panel and extendable along an inner surface of the front panel between the front panel and the front of the baby's lower torso when worn by the baby; positioning the back panel along the rear of the baby's lower torso; positioning the front panel along the front of the baby's lower torso; folding the absorbent sheath to extend along the inner surface of the front panel between the front panel and the front of the baby's lower torso; and coupling the front waist region with the back waist region to secure the diaper on the baby.

It is thus one feature of one embodiment of the present invention to quickly place a diaper with an absorbent sheath on a baby where the absorbent sheath is able to absorb urine that may escape through the top end of the front of the diaper.

These particular objects and advantages may apply to only some embodiments falling within the claims and thus do not define the scope of the invention.

### BRIEF DESCRIPTION OF THE DRAWINGS

FIG. 1 is a simplified front perspective view of a diaper of the present invention having an absorbent flap extending outward and upward at the front panel of the diaper, and an inset showing the layers of absorbent material of the diaper;

FIG. 2 is a simplified rear perspective view of the diaper of FIG. 1 showing the absorbent flap tucked inside the front of the diaper, for example, as it is being worn by a baby, to prevent liquid leaks out through the top front end of the diaper at the baby's waist;

FIG. 3 is a simplified front perspective view of an alternative embodiment of the present invention having an absorbent flap attached to the outer surface of the front panel of the diaper; and

FIG. 4 is a simplified rear perspective view of an alternative embodiment of the present invention having an absorbent flap attached to the inner surface of the front panel of the diaper.

### DETAILED DESCRIPTION OF THE PREFERRED EMBODIMENT

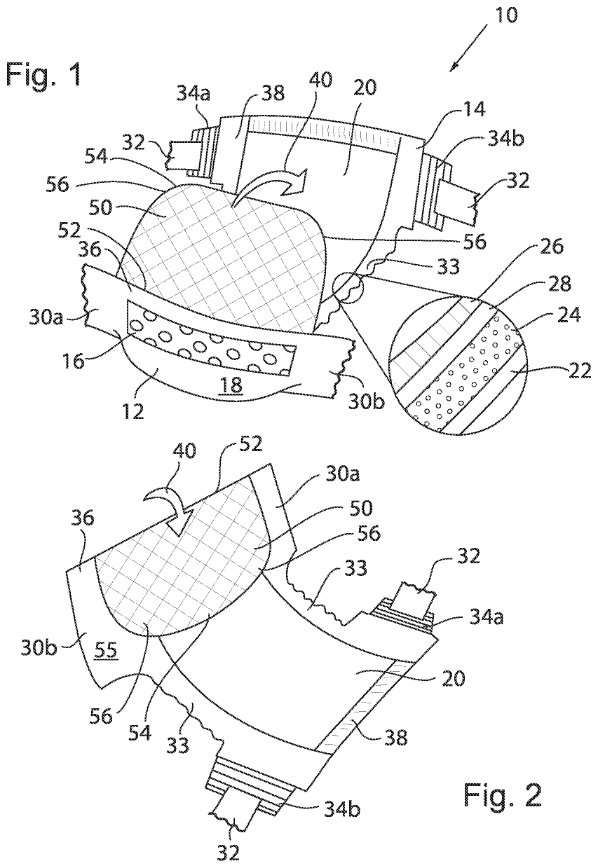

Referring now to FIG. 1, a baby's diaper 10 is shown from a front end having a front side panel 12 connected to a back side panel 14. The front side panel 12 of the diaper 10 is generally positioned to extend along a front side (anterior) of a baby's lower torso while the back side panel 14 of the diaper 10 is generally positioned to extend along a back side (posterior) of the baby's lower torso.

Prior to placement of the diaper 10 on the baby, the front side panel 12 of the diaper 10 may be easily identifiably and distinguished from the back side panel 14 by a colorful frontal tape 16 on a front surface 18 of the front side panel 12 displaying a colorful graphic, print, pattern, or design which visually designates the front side panel 12.

The front side panel 12 and the back side panel 14 may be opposed ends of a diaper substrate 20 of the baby's diaper 10. The diaper substrate 20 may be a generally rectangular pad and includes multiple layers of material (as seen in the inset of FIG. 1) extending between the front side panel 12 to the back side panel 14 along a sagittal axis of the baby. The diaper substrate 20 may extend from a front side (anterior) of the baby to a back side (posterior) of the baby in order to catch and absorb waste products.

The multiple layers of the diaper substrate 20 assist with the absorption of liquid on the inside of the diaper 10 and block moisture from leaving the inside of a worn diaper 10. The layers of the diaper substrate 20 may include an outermost layer that is exposed to the environment, an interior absorbent layer, an acquisition distribution layer, and an innermost layer that contacts the baby's skin.

In one embodiment of the present invention, a waterproof outer layer 22 forms an outermost layer of the diaper substrate 20 and is generally exposed to an exterior environment. The waterproof outer layer 22 may be a water-resistant or waterproof material or film that prevents wetness from passing through the outermost layer and ensures that there is no leakage from the inside of the diaper 10 to the exterior of the diaper 10. The waterproof outer layer 22 may be a breathable material or film, allowing water vapor and air to pass through the layer but not liquids, for increased skin comfort. In one embodiment of the present invention, the waterproof outer layer 22 may be a hydrophobic nonwoven film such as a polyethylene (PE) film or a non-woven laminated material with polyethylene (PE) or polypropylene (PP) film.

Again, the waterproof outer layer 22 may include printed designs and graphics, for example, decorating the entire exterior of the diaper 10 and/or supporting the colorful frontal tape 16 on the front side panel 12 visually indicating the front side of the diaper 10.

Next, an interior layer behind the waterproof outer layer 22, is an absorbent core 24 that absorbs the fluids and traps the liquid within the interior layer. In one embodiment of the present invention, the absorbent core 24 may be a matrix composed of (a) a fibrous material and (b) a superabsorbent polymer (SAP). The fibrous material has good liquid absorbing capacity and may be wood pulp fluff material or air-laid synthetic fibers. The SAPs may be fine granules that increase the retention capacity and have quick liquid absorption speed, high pressurized absorption, good strength, and good moisture resistance. In one embodiment of the present invention, the SAP are sodium polyacrylates granules.

Next, an inner sheet 26 forms an innermost layer of the diaper substrate 20 and contacts the baby's skin to quickly transfer liquids to the absorbent core 24 while remaining soft and dry so that it does not irritate the baby's skin. Some inner sheets 26 include a skin care lotion which protects the skin from over hydration and reduces irritation. The inner sheet 26 is liquid permeable to allow urine to quickly reach the absorbent core 24. In one embodiment of the present invention, the inner sheet 26 may be a hydrophilic nonwoven material such as a polypropylene (PP) film.

In some embodiments, sandwiched between the absorbent core 24 and the inner sheet 26 may be an acquisition distribution layer 28 which moves the liquid away from the baby's skin and distributes it evenly across the diaper substrate 20 for improved absorbency. The acquisition distribution layer 28 is particularly useful in thinner diapers. In one embodiment of the present invention, the acquisition distribution layer 28 may be trough air bond (TAB) nonwoven material, an aperture sheet (i.e., made of perforated plastic film), or resin bonded nonwoven material.

Referring now to FIGS. 1 and 2, a fastening system of the diaper 10 allows the diaper 10 to be comfortably fastened around the baby's waist. The front side panel 12 of the diaper 10 may include front waist straps 30a , 30b extending laterally outwardly, in left and right directions, from a top end 36 of the front side panel 12. The front waist straps 30a , 30b are received by fasteners 32 of the back side panel 14 as further described below.

The back side panel 14 of the diaper 10 may include stretch side panels 34a , 34b which allow the fasteners 32 to extend laterally outwardly, in left and right directions, from a top end 38 of the back side panel 14. The fasteners 32 and the stretch side panels 34a , 34b can stretch toward the front waist straps 30a , 30b to connect the top end 38 of the back side panel 14 to the top end 36 of the front side panel 12. The stretch side panels 34a , 34b may be an elastic material such as polyurethane or polyester foam elastics or synthetic rubber or Lycra/Spandex elastics. The fasteners 32 may be releasable adhesive tape, hook and loop tape (Velcro), snaps and the like which allows the back side panel 14 and the front side panel 12 to be easily coupled and decoupled to easily put on and take off the diaper 10.

The diaper 10 may include diaper fit features which provide leak guards around the baby's legs and around the baby's waist. Extending along the left and right sides of the diaper 10 are leg cuff areas 33 which hug the baby's legs in order to provide comfort but also prevent leaks. The leg cuff areas 33, stretch side panels 34a , 34b , and/or the front waist straps 30a , 30b may use an elastic material to improve the fit of the diaper 10 around the baby's legs and waist and to prevent leakage. The top end 36 of the front side panel 12 and the top end 38 of the back side panel 14 may also include an elastic material for stretch and comfort. The elastic material may be polyurethane or polyester foam elastics, or synthetic rubber or Lycra/Spandex elastics. The diaper 10 is desirably pulled snug against the baby's torso to prevent leakages around the baby's waist and around the baby's legs.

The diaper 10 may be as generally described in U.S. Pat. No. 6,554.816 entitled “Absorbent articles with shaped fastening component”; U.S. Pat. No. 6,645,190 entitled “Absorbent article with non-irritating refastenable seams”; U.S. Pat. No. 6,849,067 entitled “Absorbent articles with refastenable side seams” and U.S. Pat. No. 6,764,475 entitled “Absorbent articles having differential strength refastenable seam”; each of which are hereby incorporated by reference.

Still referring to FIGS. 1 and 2, the top end 36 of the front side panel 12 may include an absorbent sheath or flap 50 extending outwardly and upwardly from the top end 36 of the front side panel 12. The absorbent flap 50 has an attached end 52 coupled to the top end 36 of the front side panel 12 opposite a free end 54 that is unattached. The absorbent flap 50 may bend or fold at the attached end 52 to fold over the top end 36 (as shown by arrow 40) and extend downward along a rear surface 55 of the front side panel 12 (as seen in FIG. 2).

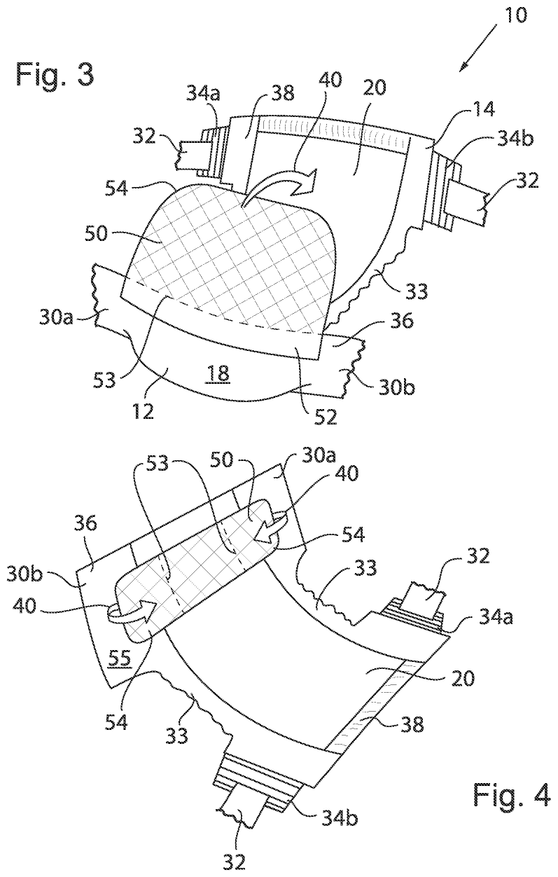

Referring to FIG. 3, in an alternative embodiment of the present invention, the absorbent flap 50 may be attached to the front surface 18 of the front side panel 12 below the top end 36 of the front side panel 12 and further include a crease or indentation 53 which allows the absorbent flap 50 to fold at the top end 36 of the front side panel 12 to extend downward along the rear surface 55 of the front side panel 12. In this respect, the absorbent flap 50 may be attached to the front side panel 12 on the outside of the diaper 10 and may extend over and fold at the top end 36 of the front side panel 12 to provide an additional absorbent material at the top end 36.

Referring to FIG. 4, in an alternative embodiment of the present invention, the absorbent flap 50 may be attached to the rear surface 55 of the front side panel 12 below the top end 36 of the front side panel 12 and further include creases or indentations 53 which allow it to be folded away from the rear surface 55 of the front side panel 12. For example, the upper edge of the absorbent flap 50 may be attached to the inner rear surface 55 to allow the absorbent flap 50 to extend downward along the rear surface 55 and allowing the lower edge to be an unattached free end 54, or the lower edge of the absorbent flap 50 may be attached to the inner rear surface 55 to allow the absorbent flap 50 to extend upward along the rear surface 55 and allowing the upper edge to be an unattached free end 54, or the outer lateral left and right edges of the absorbent flap 50 may be attached to the inner rear surface 55 of the front side panel 12 and allowing the center to be unattached, or a center of the absorbent flap 50 may be attached to the inner rear surface 55 of the front side panel 12 and allowing the outer lateral left and right edges to be unattached free ends 54 (as shown in FIG. 4). In this respect, the absorbent flap 50 may be attached to the front side panel 12 on the inside of the diaper 10 and may fold on the inside of the diaper 10 to provide an additional absorbent material on the inside of the diaper.

The absorbent flap 50 may be a rectangular, oval, circular, or semi-circular pad. The attached end 52 of the absorbent flap 50 may have a straight edge and the free end 54 may have curved outer edges 56. The curved outer edges 56 may prevent the absorbent flap 50 from contacting or interfering with the baby's legs when folded to the inside of the diaper 10. It is understood that the absorbent flap 50 may be any contemplated shape or size.

The absorbent flap 50 may extend substantially a full width of the top end 36 of the front side panel 12 in order to provide an additional barrier to leaks across the top end 36 of the front side panel 12. The absorbent flap 50 may have a width extending along the top end 36 of the front side panel 12 that is between 3 and 6 inches and at least 3 inches and at least 4 inches and at least 6 inches and approximately 4 inches.

The absorbent flap 50 may have a length that extends from the attached end 52 to the free end 54, perpendicular to the top end 36 of the front side panel 12. The length of the absorbent flap 50 may be at least one-third a length of the front side panel 12 and at least one-half a length of the front side panel 12. The length of the absorbent flap 50 may be between 2 and 4 inches and at least 2 inches and at least 3 inches and approximately 3 inches.

In some embodiments the length of the absorbent flap 50 is made slightly longer, for example, an additional 1 to 3 inches longer, so that the absorbent flap 50 can be attached to the front side panel 12 below the top end 36 and folded at the top end 36 at a crease or indentation, as described above with respect to the alternative embodiment.

It is understood that the length and width of the absorbent flap 50 will depend on the size of the diaper 10 which varies with the size of the baby.

The absorbent flap 50 may include the same layers and respective materials as the diaper substrate 20 described above, for example, the waterproof outer layer 22, the absorbent core 24, inner sheet 26, and acquisition distribution layer 28 described above.

In one embodiment of the present invention, the absorbent flap 50 may include (a) a fibrous material such as wood pulp fluff material or air-laid synthetic fibers and (b) a super absorbing polymer (SAP) such as sodium polyacrylates granules. It is understood that the absorbent flap 50 may include other types of known absorbent material, for example, commonly found in absorbent diaper liners and pads. Examples of absorbent materials include absorbent foam (flex foam) or absorbent wood cellulose with absorbent gel, rayon, or polyester. Other materials used in the waterproof outer layer 22 and inner sheet 26 may include polyolefins and petrolatum and zinc oxide (ingredients found in skin lotions). The absorbent flap 50 may also take a quilted pattern.

The absorbent flap 50 may be desirably thinner than the diaper substrate 20. In one embodiment of the present invention, the absorbent flap 50 has a thickness of less than 0.3 inches and less than 0.2 inches and less than 0.1 inches.

Referring now to FIG. 2, in the process of putting the diaper 10 on the baby, the absorbent flap 50 is folded at the top end 36 of the front side panel 12, for example, at the attached end 52 or at a crease or indentation, to allow the absorbent flap 50 to be positioned inside the diaper 10 and extended along the inner rear surface 55 of the front side panel 12. It may be desired for the absorbent flap 50 to be tucked inside the diaper so that it surrounds the baby boy's penis so that urine is directed up into the absorbent flap 50 or between the inner rear surface 55 and the absorbent flap 50. The absorbent flap 50 may be folded at the top end 36 and tucked inside the diaper 10 before, during, or after the fasteners 32 of the back side panel 14 are secured to the waist straps 30a , 30b of the front side panel 12.

Referring now to FIG. 3, in an alternative embodiment of the present invention, for example, when the absorbent flap 50 is attached to the front surface 18 of the front side panel 12 below the top end 36 of the front side panel 12, the absorbent flap 50 is folded over the top end 36 of the front side panel 12, for example, at a crease or indentation 53, to allow the absorbent flap 50 to be positioned inside the diaper 10 and extended along the inner rear surface 55 of the front side panel 12. It may be desired for the absorbent flap 50 to be tucked inside the diaper so that it surrounds the baby boy's penis so that urine is directed up into the absorbent flap 50 or between the inner rear surface 55 and the absorbent flap 50. The absorbent flap 50 may be folded over the top end 36 and tucked inside the diaper 10 before, during, or after the fasteners 32 of the back side panel 14 are secured to the waist straps 30a , 30b of the front side panel 12.

Referring now to FIG. 4, in an alternative embodiment of the present invention, for example, when the absorbent flap 50 is attached to the inside of the diaper 10 such that it extends along the inner rear surface 55 of the front side panel 12 without having to be folded over the top end 36 and tucked inside the diaper 10, the absorbent flap 50 is folded on the inside of the diaper so that it surrounds the baby boy's penis and urine is directed into the absorbent flap 50. For example, the free ends 53, which may be the upper edge, lower edge, center portion, or outer lateral edges of the absorbent flap 50, may be folded away from the inner rear surface 55 to wrap around the baby boy's penis. The absorbent flap 50 may be folded to surround the baby boy's penis before, during, or after the fasteners 32 of the back side panel 14 are secured to the waist traps 30a , 30b of the front side panel 12.

It is contemplated that multiple absorbent flaps 50 may be installed on the diaper 10. It is also contemplated that an absorbent flap 50 may also be installed on the back side panel 14 in a similar manner as the front side panel 12 to prevent leaks at the back side of the baby. For example, an absorbent flap 50 may also be attached to the back side panel 14 in a similar manner as described above and the absorbent flap 50 may bend or fold over the top end 38 to extend downward along the rear surface 55 of the back side panel 14.

It is understood that the absorbent flap 50 may be used to prevent the flow of excess urine out through the top of the diaper 10 at the top end 36 which may result from the upward direction of urine flow, or the flow of excess liquid to gaps around the baby's waist, for example when the baby is laying prone. The absorbent flap 50 catches and absorbs the urine to prevent leakages at the top end of the diaper 10.

Certain terminology is used herein for purposes of reference only, and thus is not intended to be limiting. For example, terms such as “upper,” “lower,” “above,” and “below,” refer to directions in the drawings to which reference is made. Terms such as “front,” “back,” “rear,” “bottom,” and “side,” describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first,” “second,” and other such numerical terms referring to structures do not imply a sequence or order unless clearly indicated by the context.

When introducing elements or features of the present disclosure and the exemplary embodiments, the articles “a,” “an,” “the,” and “said,” are intended to mean that there are one or more of such elements or features. The terms “comprising”, “including,” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

What I claim is:

1. A disposable diaper to be worn by a human baby and extending along a sagittal axis of the baby when worn, the diaper comprising:
an absorbent pad extending along the sagittal axis having at least one layer;
a front panel extending from a front end of the absorbent pad and configured to extend along a front of a baby's lower torso and supporting a front waist region configured to extend along a baby's waist;
a back panel extending from a rear end of the absorbent pad and configured to extend along a rear of a baby's lower torso and supporting a back waist region configured to extend along a baby's waist and to couple to the front waist region; and
an absorbent sheath extending outwardly from the front panel and extendable along an inner surface of the front panel between the front panel and the front of the baby's lower torso when worn by the baby.

2. The diaper of claim 1 wherein the absorbent sheath is thinner than the absorbent pad, thickness measured along a longitudinal axis perpendicular to the sagittal axis.

3. The diaper of claim 1 wherein the absorbent sheath has a thickness that is less than 0.3 inches, the thickness measured along a longitudinal axis perpendicular to the sagittal axis.

4. The diaper of claim 1 wherein the absorbent sheath comprises an absorbent layer configured to absorb and trap liquid.

5. The diaper of claim 4 wherein the absorbent layer comprises at least one of a fibrous material and a super absorbing polymer.

6. The diaper of claim 4 wherein the absorbent sheath comprises a contacting layer that is configured to contact the baby's lower torso and is permeable to liquids.

7. The diaper of claim 1 wherein the absorbent sheath is a flap attached to the front panel and is foldable downwardly toward a rear end of the front panel.

8. The diaper of claim 1 wherein the absorbent sheath extends substantially an entire width of a top edge of the front panel.

9. The diaper of claim 1 wherein the absorbent sheath has a width of at least 3 inches.

10. The diaper of claim 9 wherein the absorbent sheath has a width of at least 4 inches.

11. The diaper of claim 1 wherein the absorbent sheath extends at least one third a length of the front panel, the length measured along a longitudinal axis perpendicular to the sagittal axis.

12. The diaper of claim 1 wherein the absorbent sheath has a length of at least 2 inches.

13. The diaper of claim 12 wherein the absorbent sheath has a length of at least 3 inches.

14. The diaper of claim 1 wherein the absorbent sheath is substantially rectangular with curved edges.

15. The diaper of claim 1 wherein the at least one layer of the absorbent substrate comprises an innermost layer configured to contact the baby's lower torso and is permeable to liquids, an interior layer configured to absorb and trap liquid, and an outermost layer configured to be exposed to an environment and is impermeable to liquid.

16. The diaper of claim 1 wherein an outer surface of the front panel includes a graphic design.

17. The diaper of claim 1 further comprising fasteners extending outwardly from the back waist region of the rear side panel.

18. The diaper of claim 17 wherein the fasteners include an adhesive.

19. The diaper of claim 1 wherein the front waist region and back waist region are elastic.

20. A method of putting an absorbent article on a baby comprising:
providing a disposable diaper having an absorbent pad extending along a sagittal axis of the baby having at least one layer, a front panel extending from a front end of the absorbent pad and configured to extend along a front of a baby's lower torso and supporting a front waist region configured to extend along a baby's waist, a back panel extending from a rear end of the absorbent pad and configured to extend along a rear of a baby's lower torso and supporting a back waist region configured to extend along a baby's waist and to couple to the front waist region, and an absorbent sheath extending outwardly from the front panel and extendable along an inner surface of the front panel between the front panel and the front of the baby's lower torso when worn by the baby;
positioning the back panel along the rear of the baby's lower torso;
positioning the front panel along the front of the baby's lower torso;
folding the absorbent sheath to extend along the inner surface of the front panel between the front panel and the front of the baby's lower torso; and
coupling the front waist region with the back waist region to secure the diaper on the baby.
